(19) United States
(12) Patent Application Publication
(10) Pub. No.: US 2006/0127290 A1
(43) Pub. Date: Jun. 15, 2006

(54) Packaging machine with multiple UV lamps transverse to package path

(75) Inventors: Bengt Bengtsson, River Falls, WI (US); Jenifer Hemphill, Woodbury, MN (US); Kenneth Millard Eno, Woodville, WI (US)

Correspondence Address: TETRA PAK US INTELLECTUAL PROPERTY DEPARTMENT;TETRA PAK INC., 101 COPORATE WOODS PARKWAY, VERNON HILLS, IL, 60061, US

(73) Assignee: Tetra Laval Holdings & Finance, S.A., Pully (CH)

(21) Appl. No.: 11/011,259

(22) Filed: Dec. 14, 2004

(51) Int. Cl. A61L 2/16; A61L 2/10

(52) U.S. Cl. 422/304; 422/24; 422/28

(57) ABSTRACT

An ultraviolet sterilization system for a form, fill and seal packaging machine, includes at least three ultraviolet lamps, each having an elongated dimension and a transverse dimension. Each lamp is mounted within a housing having a reflector. The lamps are mounted adjacent one another in a side-by-side manner such that the elongated dimensions are parallel to one another and transverse to the package conveyance path. The ultraviolet sterilization system, in conjunction with a hydrogen peroxide application at a concentration of about 3.0 percent, provides a log reduction of at least about 4.5 to 5.0 of microbial colony forming units.

### BACKGROUND OF THE INVENTION

The present invention is directed to an ultraviolet (UV) sterilization system for a packaging machine. More particularly, the present invention is directed to a multi-lamp sterilization system for form, fill and seal packaging machines.

Form, fill and seal packaging machine are well known in the art, as are sterilization systems for use with such machines. These machines are widely used in the food packaging industry for forming a package, filling the package with a liquid or solid food (or a mixture of liquid and solid foods), and sealing the package after filling.

In such machines, sterilization systems are in place to eradicate (i.e., kill) microbes such as bacteria, yeast and mold. Commonly, the sterilization systems are directed to sterilizing the packages before and/or after forming the package, prior to filling and sealing.

Different types of sterilization systems are also used. For example, one type of sterilization system uses hydrogen peroxide as a liquid or in vapor form that is sprayed onto the exterior of the packages and/or into the interior of the packages. The hydrogen peroxide concentration can vary from about 3 percent to as much as 35 percent in order to achieve the high log reduction factor (bacterial kill rate) demands. Other types of sterilization systems use energy in the form of, for example, ultraviolet emissions or electron beam emissions to kill these microbes. Still other types of sterilization systems use combinations of these systems and methods.

Prevailing sterilization systems use hydrogen peroxide that is applied directly to the packaging generally at a sterilization station within the machine. Exemplary arrangements for machines and machine sterilizations systems, methods and the like are disclosed in Cicha, et al., U.S. Pat. No. 6,406,666, Palaniappan, et al., U.S. Pat. No. 6,120,730, Lees, U.S. Pat. No. 6,058,678, Palaniappan, et al., U.S. Pat. No. 6,056,918 and Eno, U.S. Pat. No. 5,809,740, all of which patents are commonly assigned with the present application and are incorporated herein by reference. One system that uses ultraviolet lamps has two lamps mounted transverse to the carton path, while another arrangement includes a single elongated lamp extending along or parallel to the carton path.

Although all of these systems work well and function well, there are limits to the amount of residual hydrogen peroxide (which is generally present in the form of condensed vapor remaining on the packaging material surface) that can remain on packaging when food is introduced to the package, and hydrogen peroxide that can be in the local machine environment. As such an appropriate drying time is necessary to assure that these upper limits are met. And, of course, the higher the concentration of hydrogen peroxide used (e.g., 35 percent as opposed to 3 percent), the greater the probability that a higher level of residual hydrogen peroxide will remain longer on the package. In addition, higher hydrogen peroxide concentrations can result in issues arising with respect to degradation of machine materials and the like.

Furthermore, more than just meeting the sterility or cleanliness standards (which permit some amount of microbes), it is known that greater amounts of microbial contamination will result in packaged foods that have shorter shelf lives. That is, typically, as the amount of microbial contamination increases, the shelf life of the product decreases. This is of greatest concern for long or extended shelf life products and for aseptic products where the product must meet strict commercial sterility requirements, and particularly true for high acid ambient distribution (“HAAD”) product, which allows for a high acid (pH<4.5) product such as orange juice to be stored unrefrigerated for an extended time period. Often, the carton blank is for a HAAD product has a film structure that includes an aluminum barrier

Nevertheless, for all hygiene levels, these requirements are strictly controlled and enforced by governmental regulatory agencies such as for example the United States Food and Drug Administration (USFDA) and the United States Department of Agriculture (USDA).

Accordingly, there exists a need for a sterilization system for a form, fill and seal packaging machine that increases the ability to kill or otherwise eliminate microbes that may be present on or in the packaging. Desirably, such a sterilization system uses a lower concentration of hydrogen peroxide, while maintaining a high kill rate (log reduction) of bacteria. Most desirably, such a system is compatible with the overall operation of known form, fill and seal machines.

### BRIEF SUMMARY OF THE INVENTION

A multi-lamp ultraviolet sterilization system for a form, fill and seal packaging machine provides, in conjunction with a hydrogen peroxide application at a concentration of about 3.0 percent, a log reduction of at least about 4.5 to 5.0 of microbial colony forming units. A packaging machine is configured for forming, filling and sealing packages and includes a conveying system for conveying the packages through a plurality of stations along a conveyance path. The packaging machine includes a sterilization station having a hydrogen peroxide distribution system.

The ultraviolet sterilization system includes at least three ultraviolet lamps. Each lamp has an elongated dimension and a transverse dimension, and is mounted within a housing having a reflector. The lamps are mounted adjacent one another in a side-by-side manner such that the elongated dimensions are parallel to one another and transverse to the package conveyance path.

A preferred system includes six ultraviolet lamps mounted adjacent one another in a side-by-side manner. The elongated dimension of the lamps is greater than a width of the conveyance path. Each lamp has an intensity of about 15 milliwatts per square centimeter (mW/cm²) to about 16 mW/cm².

These and other features and advantages of the present invention will be apparent from the following detailed description, in conjunction with the appended claims.

### BRIEF DESCRIPTION OF THE SEVERAL VIEWS OF THE DRAWINGS

The benefits and advantages of the present invention will become more readily apparent to those of ordinary skill in the relevant art after reviewing the following detailed description and accompanying drawings, wherein:

FIG. 1 illustrates an exemplary form, fill and seal packaging machine having a multi-lamp ultraviolet sterilization system embodying the principles of the present invention;

FIG. 2 is a bottom perspective illustration of the ultraviolet sterilization system with cartons shown in conveyance (without a conveyor), below the lamps; and

FIG. 3 is a top perspective of the ultraviolet sterilization system showing the carton chute and a portion of a carton in the chute.

### DETAILED DESCRIPTION OF THE INVENTION

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiment illustrated.

It should be further understood that the title of this section of this specification, namely, “Detailed Description Of The Invention”, relates to a requirement of the United States Patent Office, and does not imply, nor should be inferred to limit the subject matter disclosed herein.

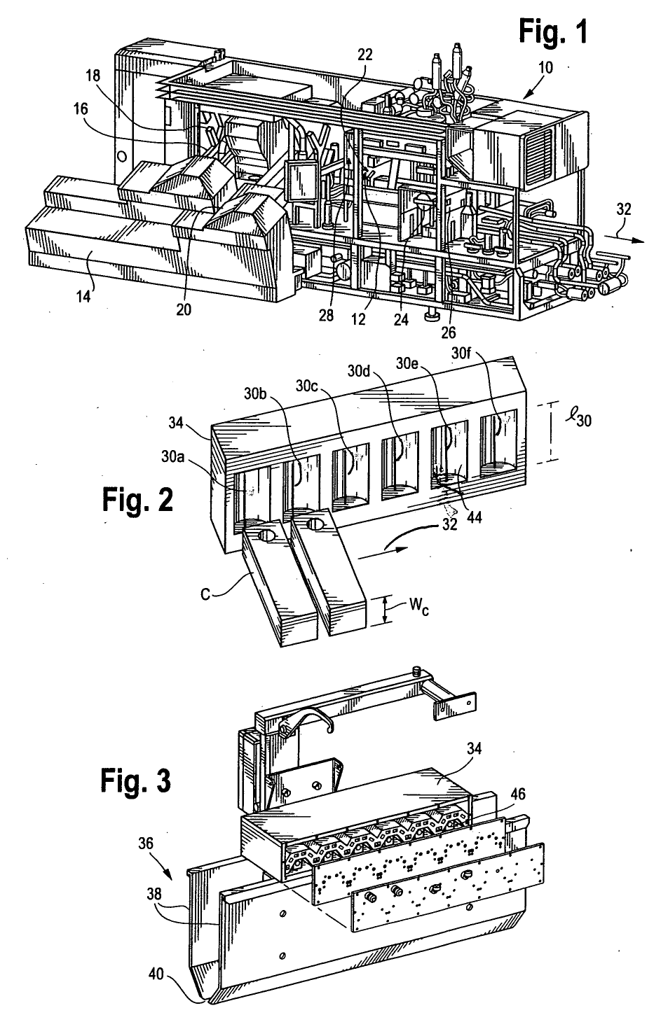

Referring now to the figures and in particular to FIG. 1, there is shown a form, fill and seal packaging machine 10 that incorporates a transverse ultraviolet irradiation system 12 for the sterilization of packages C, embodying the principles of the present invention. The form, fill and seal packaging machine 10 can be such as that disclosed in Katsumata, U.S. Pat. No. 6,012,267, which patent is assigned to the assignee of the present invention and is incorporated by reference herein. The machine 10 is configured to store, erect, fill and seal a series of cartons C moving therethrough.

A typical filling machine 10 includes a carton magazine 14 for storing the flat, folded carton blanks. The filling machine 10 includes a carton erection station 16 that receives the cartons in the flat, folded form, erects the cartons into a tubular form and seals the bottom flaps thereof. A fitment, such as the now widely recognized plastic spout (not shown), may then be applied to the partially erected carton.

In order to form the sealed bottom wall W, the tubular formed cartons are positioned on a mandrel 18 that rotates through a number of stations at which the bottom flaps are prefolded or “broken”, heated and pressed, under pressure, to one another to fuse the polymeric coating and formed the sealed bottom wall W. Following bottom wall forming, the carton is pulled from the mandrel 18 and set into an indexing conveyor 20 for sterilization, filling and top sealing at sterilization 22, filling 24 and top sealing 26 stations, respectively. In addition, on the indexing conveyor 20, closures can applied and sealed to the cartons C at a closure application station 28.

Following application of the closures, the carton C is sterilized. Typically, in order to achieve the high cleanliness standards needed for HAAD, or any extended shelf life product, a relatively high concentration hydrogen peroxide (H2O2) application (as vapor or liquid hydrogen peroxide) is used. The H2O2 concentration can be as high as 35 percent by weight of the spray. Systems are known that use H2O2 concentrations as high as about 53 percent. However, those skilled in the art will recognize and understand that while the H2O2 does in fact kill the microbes, when H2O2 concentrations are as high as 35 percent to 53 percent, the residual H2O2 remaining on the package can be above the limits set by governmental standards. These high concentration H2O2 systems can achieve log reduction rates as high as 5.9 after ten minutes of continuous machine part sterilization at a H2O2 concentration of 35 percent, as disclosed in Swank, U.S. patent application Ser. No. 10/400,305.

In a typical form, fill and seal packaging machine 10, following sterilization of the carton C (with the closure mounted thereto), the carton C is filled, at the filling station 24, with a product, such as orange juice, and the top panels are folded and sealed, at the top sealing station 26, to form the familiar gable top. The formed, filled and sealed packages are then off-loaded from the machine 10 for packaging (such as into crates) for distribution.

The present sterilization system 12 permits the use of a lower H2O2 concentration while still achieving the desired microbial kill rates. The system 12 includes a plurality of transversely oriented ultraviolet (LV) lamps 30a-f arranged in a longitudinal array along the carton indexing path 32. This configuration, although within substantially the same footprint (and thus about an equal overall “dwell time” to known systems), provides a longer directed or undiffused UV exposure to effect a higher kill rate.

A present sterilization system includes a bank of six UV lamps 30a-f with each individual lamp oriented transverse to the direction of movement of the cartons (along path 32), and with the plurality of lamps mounted adjacent one another in the longitudinal direction. In a present system, the lamps 30a-f are mounted within a single housing 34 above a chute 36 having upstanding sidewalls 38 and an open bottom end 40 (as illustrated funnel-shaped) to permit the carton drive system (e.g., the conveyor 20) to move the cartons C through the chute 36. The single housing 34 design permits the six-lamp 30a-f configuration to be incorporated into current form, fill and seal packaging machines in the same “footprint” as known UV systems, and thus without major modifications to the machine 10.

The present multi-lamp system 12 increases the UV exposure (irradiation) of the packages C within substantially the same footprint as known systems. Each UV lamp 30 is a 900 watt (W) lamp or bulb having a wavelength of about 200 to about 300 nanometers (nm). The lamps 30 each have an intensity of about 15 mW/cm² to about 16 mW/cm². The lamps are each positioned within a reflector assembly 44 having a parabolic profile to better focus the UV light emitted from the bulbs. The lamp 30 assemblies (including the lamps 30, reflectors 44 and power sources 46, as well as monitoring and local control equipment (not shown)) are cooled to maintain the lamps 30 at a desired operating temperature. In a present system, the temperature is maintained at about 15° C. to 20° C.

The elongated dimension 1₃₀ of the lamps 30 is greater than the width w_{C} of the cartons C on the conveyance path 32. This provides more “direct” UV exposure of the cartons C as they move through the machine 10. It has been found that the multi-lamp arrangement 30a-f increases the “sweet spot” of the area over which the UV irradiation is most effective. That is, the effective energy from the multi-lamp 30a-f arrangement is much greater than that available from known UV arrangements. Although this is attributed to, in part, the increased number of UV sources, it is also a result of subjecting the cartons on the path 32 to more direct UV radiation exposure. In that the present system 12 provides multiple UV sources, overall the amount of UV radiation that the carton C is subjected to is considerably greater, even though the “footprint” of the system 12 (and in particular the housing 44) is not substantially greater than that of known UV irradiation systems.

The increased effective energy has shown significantly increased microbial eradication (kill) results compared to known UV systems, using the lower (3 percent) concentration H2O2. Log reductions have been shown to increase from about 3.0 to 3.5 with the two-lamp transverse mounting arrangement and about 3.5 to 4.0 with the single longitudinal lamp arrangement to about 4.5 to 5.0 with the present multi-lamp transverse mount system 12. These are (because these are log factors), increases in kill rates approaching and in some cases exceeding a factor of ten.

All patents referred to herein, are hereby incorporated herein by reference, whether or not specifically do so within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

What is claimed is:

1. An ultraviolet sterilization system for a form, fill and seal packaging machine, the form, fill and seal packaging machine configured for forming, filling and sealing packages, the machine having a conveying system for conveying the packages through a plurality of stations along a conveyance path, the packaging machine including a sterilization station having a hydrogen peroxide distribution system, the ultraviolet sterilization system comprising:
at least three ultraviolet lamps, each lamp having an elongated dimension and a transverse dimension, each lamp being mounted within a housing having a reflector, the lamps being mounted adjacent one another in a side-by-side manner such that the elongated dimensions are parallel to one another and transverse to the package conveyance path, wherein the ultraviolet sterilization system, in conjunction with a hydrogen peroxide application at a concentration of about 3.0 percent provides a log reduction of at least about 4.5 to 5.0 of microbial colony forming units.

2. The ultraviolet sterilization system in accordance with claim 1 including six ultraviolet lamps mounted adjacent one another in a side-by-side manner.

3. The ultraviolet sterilization system in accordance with claim 1 wherein an elongated dimension of the lamps is greater than a width of the conveyance path.

4. The ultraviolet sterilization system in accordance with claim 1 wherein each lamp has an intensity of about 15 mW/cm² to about 16 mW/cm².

5. A form, fill and seal packaging machine, having an ultraviolet sterilization system, comprising:
a carton magazine;
a carton erection station;
a carton bottom sealing station;
an ultraviolet sterilization system including at least three ultraviolet lamps, each lamp having an elongated dimension and a transverse dimension, each lamp being mounted within a housing having a reflector, the lamps being mounted adjacent one another in a side-by-side manner such that the elongated dimensions are parallel to one another and transverse to the package conveyance path, wherein the ultraviolet sterilization system, in conjunction with a hydrogen peroxide application at a concentration of about 3.0 percent provides a log reduction of at least about 4.5 to 5.0 of microbial colony forming units;
a filling station; and
a top sealing station.

6. The form, fill and seal packaging machine in accordance with claim 5 including six ultraviolet lamps mounted adjacent one another in a side-by-side manner.

7. The form, fill and seal packaging machine in accordance with claim 5 wherein an elongated dimension of the lamps is greater than a width of the conveyance path.

8. The form, fill and seal packaging machine in accordance with claim 5 wherein each lamp has an intensity of about 15 mW/cm² to about 16 mW/cm².
